(19) United States
(12) Patent Application Publication
(10) Pub. No.: US 2002/0049184 A1
(43) Pub. Date: Apr. 25, 2002

(54) Solution of galactomannans as a sieving matrix in capillary electrophoresis

(76) Inventors: Vladislav Dolnik, Mountain View, CA (US); William A. Gurske, Fremont, CA (US); Allan Padua, San Mateo, CA (US)

Correspondence Address: AMERSHAM PHARMACIA BIOTECH INC., PATENT DEPARTMENT, P O BOX 1327, 800 CENTENNIAL AVENUE, PISCATAWAY, NJ, 088551327

(21) Appl. No.: 09/946,396

(22) Filed: Sep. 5, 2001

Related U.S. Application Data
(60) Provisional application No. 60/230,507, filed on Sep. 6, 2000.
(60) Provisional application No. 60/230,508, filed on Sep. 6, 2000.

(51) Int. Cl. A61K 31/736; C08B 37/00

(52) U.S. Cl. 514/54; 536/114

(57) ABSTRACT

Galactomannans with a reduced protein content in an anhydrous, powder form, a process of producing same and a capillary column containing a composition of the galactomannans for use as a sieving matrix in capillary electrophoresis are disclosed.

### CROSS-REFERENCE TO RELATED APPLICATIONS

This application claims priority to U.S. Provisional Application Ser. Nos. 60/230,507 and 60/230,508, filed on Sep. 6, 2000, the entire disclosure of which is incorporated herein.

### BACKGROUND OF THE INVENTION

1. Field of the Invention

The present invention refers to use of galactomannans including guar gum, tara gum, and locust bean gum as a sieving matrix in separation methods such as electrophoresis and chromatography and to modifying surfaces. Specifically, the object of the invention is the use of galactomannans as a sieving matrix in capillary electrophoresis.

2. Description of Related Art

Electrophoresis in sieving matrices is a powerful method for separation of biopolymers including nucleic acids [Cohen, A. S. et al., Proc. Natl. Acad. Sci. U.S.A. 1988, 85, 9660, Salas-Solano, O. et al., Anal. Chem. 1998, 70, 3996, Dovichi, N. J., Electrophoresis 1997, 18, 2393, Fung, E. N. and Yeung, E. S., Anal. Chem. 1995, 67, 1913, Marsh, M. et al., J. Cap. Electrophoresis. 1997, 4, 83, Bashkin, J. et al., J. Capillary Electrophoresis 1996, 3, 1], proteins [Cohen, A. S. and Karger, B. L., J. Chromatogr. 1987, 397, 409, Dolnik, V. et al., J. Microcol. Sep. 1991, 3, 155, Ganzler, K. et al., Anal. Chem. 1992, 64, 2665, Widhalm, A. et al., J. Chromatogr. 1991, 549, 446], polyamino acids [Dolnik, V. and Novotny, M., Anal. Chem. 1993, 665, 563, Dolnik, V. et al., Biopolymers, 1993, 33, 1299], and polysaccharides [Liu, J. et al., Anal. Chem. 1992, 64, 1328, Hong, M. F. et al., Anal. Chem. 1998, 70, 568].

In DNA sequencing, which is an important application of capillary electrophoresis, hydroxyethyl cellulose [Bashkin, J. et al., J. Cap. Electrophoresis 1996, 3, 1], polyethylene oxide {Fung, E. N., Yeung, E. S. Anal. Chem. 1995, 67, 1913] polydimethylacrylamide [Madabhushi, R. S., Electrophoresis 1998, 19, 224], polyvinyl pyrrolidone [Gao, Q. F. and Yeung, E. S., Anal. Chem. 1998, 70, 1382], and linear polyacrylamide [Ruiz-Martinez, M. C. et al., Anal. Chem. 1993, 65, 2851, Best, N., et al., Anal. Chem. 1994, 66, 4063, Goetzinger, W. et al., Electrophoresis 1998, 19, 242] are the most frequently used sieving polymers. Good performance has been so far demonstrated for linear polyacrylamide, that produced DNA sequencing read lengths over 1300 bases within two hours [Zhou, H. H. et al., Anal. Chem. 2000, 72, 1045].

There are several types of galactomannans, a class of linear polysaccharides with a backbone (1→4) linked β-D-mannopyranosyl units that have side stubs of (1→6) -linked α-D-galactopyranosyl groups. The most important galactomannans are: locust bean gum, tara gum, guar gum (guaran), and fenugreek gum. These galactomannans differ by the frequency, with which the galactosyl groups are attached to the mannose backbone. The ratio of D-mannosyl to D-galactosyl units is 3.8:1 for locust bean gum, approx. 3:1 for tara gum, 1.8:1 for guar gum, and about 1:1 for fenugreek gum. Locust bean gum, guar gum, and tara gum are produced commercially and have various applications in food industry and as an additive to fracturing fluids in petroleum industry [Maier, H. et al., in: Whistler, R. L., BeMiller, J. N. (Eds.), Industrial Gums. Polysaccharides and Their Derivatives, Academic Press, San Diego 1993, p. 181].

Only limited number of methods have been applied to purify guaran and other galactomannans. Usually guaran is dissolved in water and a cold water insoluble pellet is removed by filtration or centrifugation. To separate guaran from proteins, the precipitation with alcohols including ethanol [Heine, E. and Whistler, L. R., J. Am. Chem. Soc. 1948, 70, 2249, Robinson, G. et al., Carbohydr. Res. 1982, 107, 17, Ellis, P. R. and Morris, E. R., Diabetes Res. 1991, 18, 378, Maruyama, K. et al., European Patent 732342, 1996, Gebert, M. S. and Friend, D. R., Pharm. Dev. Technol. 1998, 3, 315, Gebert M. S. et al., U.S. Pat. No. 6,063,402], methanol [Morikawa, M. et al., Japanese Patent 63101402 JP, 1988], and isopropyl alcohol [Ninomiya, H. et al., Japanese Patent 630035606 JP, 1988, Lopes, D. A. et al., Food Hydrocolloids 1990, 4, 277] have been used. The protein content stays relatively high (above 0.1%). Phenol extraction of trace proteins was applied as well [Chandrasekaran, R. et al., Carbohydrate Res. 1998, 306, 243]. More sophisticated technique was used to purify galactomannan from seeds of Poinciana Pulcherrima L. by affinity chromatography on immobilized jackfruit pectin. This method can produce only limited quantities of purified galactomannan [Chowdhury, B. and Chatterjee, B. P., Indian J. Chem. Sect. B Org. Chem. Incl. Med. Chem. 1987, 26, 637.].

The high viscosity of diluted galactomannan solutions indicates that they may be a useful sieving matrix in capillary electrophoresis of biopolymers. This has been shown by the use of Synergel®, a low molecular mass locust bean gum for separation of restriction fragments of DNA [Dolnik, V. and Novotny, M., J. Microcol. Sep. 1992, 4, 515]. Guaran was used even as a dynamic wall coating for separation of proteins by capillary electrophoresis at low pH. The authors intentionally worked at guaran concentrations about 0.1% to avoid sieving effect of entangled solution of guaran [Liu, Q. et al., Chromatographia 1998, 47, 219]. Using crude guaran as a sieving matrix with a good separation performance is impossible due to presence of impurities namely proteins that can interact with separands and deteriorate the separation.

As the above discussion suggests, improvements in galactomannan purification methods, in terms of increasing the purity of product by reducing the protein content, are desirable. This concern is addressed in greater detail below.

### SUMMARY OF THE INVENTION

Accordingly, it is the object of the invention to provide galactomannans with a reduced protein content preferably in an anhydrous, powder form and a process of producing them, as well as, a capillary column containing a composition of the galactomannans for use as a sieving matrix in capillary electrophoresis. This objective was met by the present invention, which relates in one aspect to a composition. The composition comprises a galactomannan having 50-90% by weight of mannose residues, 10-50% by weight of galactose residues and less than 0.1% by weight of protein. In one embodiment the galactomannan is guar gum of Cyamopsis tetragonoluba. In a second embodiment the galactomannan is tara gum of Caesalpinia spinosa. In a third embodiment the galactomannan is locust bean gum of Ceratonia siliqua. In another embodiment the galactomannan has a weight molecular mass of 3×10⁵ to 4×10⁶.

In another aspect, the invention relates to a process of purifying galactomannans. The process comprises treating the galactomannan with a anion exchanger, treating the galactomannan with an cation exchanger, and precipitating the galactomannan with an organic solvent selected from the group consisting of ethers, esters, ketones and aldehydes. In one preferred embodiment the organic solvent is acetone.

In another preferred embodiment, the process further comprises treating the galactomannan with a diluted aqueous solution of base. In one embodiment, the base is an alkali hydroxide, selected from the group consisting of sodium hydroxide, potassium hydroxide and lithium hydroxide. In another embodiment, the alkali hydroxide is in the concentration of 0.001 M to 1.0 M.

In another embodiment, the steps of treating the galactomannan with a cation and an anion exchanger are performed by ion exchange chromatography. In another embodiment the same treating steps are performed by mixing and stirring the galactomannan with ion exchange resins. In another embodiment one or both of said anion exchange resin and said cation exchange resin is a polystyrene resin. In another embodiment and one or both of said anion exchange resin and said cation exchange resin is a dextran crosslinked resin. In a further embodiment the anion exchanger resin is QAE Sephadex or Source 30Q. In yet a further embodiment the cation exchanger resin is SP Sephadex or Source 30S. In another preferred embodiment, the process further comprises treating the galactomannan with a mixed ion exchanger, such as Amberlite MB-150 to deionize it and remove salts.

In another aspect, the invention relates to a capillary column. The capillary column comprises a capillary electrophoresis tube and contained within said tube, a composition comprising an aqueous solution containing 0.5 to 3.0% by weight of a galactomannan according to claim 1, a buffer having a concentration of 0.01 M to 0.1 M, a denaturant having a concentration of 0.0 M to 8.0 M, and a detergent having a concentration of 0.0 to 1.0% by weight. In one embodiment the galactomannan is guar gum of Cyamopsis tetragonoluba. In a second embodiment the galactomannan is tara gum of Caesalpinia spinosa. In a third embodiment the galactomannan is locust bean gum of Ceratonia siliqua. In another embodiment the galactomannan has a weight molecular mass of 3×10⁵ to 4×10⁶. In a further embodiment said detergent is selected from the group consisting of sodium dodecyl sulphate, lithium dodecyl sulphate, cetyltrimethylammonium bromide, cetyltrimethylammonium chloride, pyridinium bromide, pyridinium chloride, and carboethopendecenium bromide.

The above objects and features of the invention will become more fully apparent when the following detailed description of the invention is read in conjunction with the accompanying drawings.

### BRIEF DESCRIPTION OF THE DRAWINGS

FIG. 1 is a capillary electropherogram of a protein extract from crude guar gum.

FIG. 2 is a capillary electropherogram of a protein extract from crude locust bean gum.

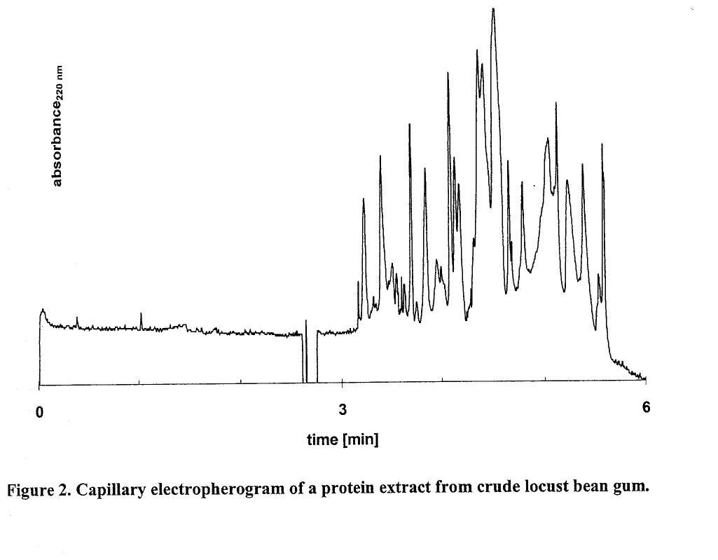

FIG. 3 is capillary electropherogram of a protein extract from crude tara gum.

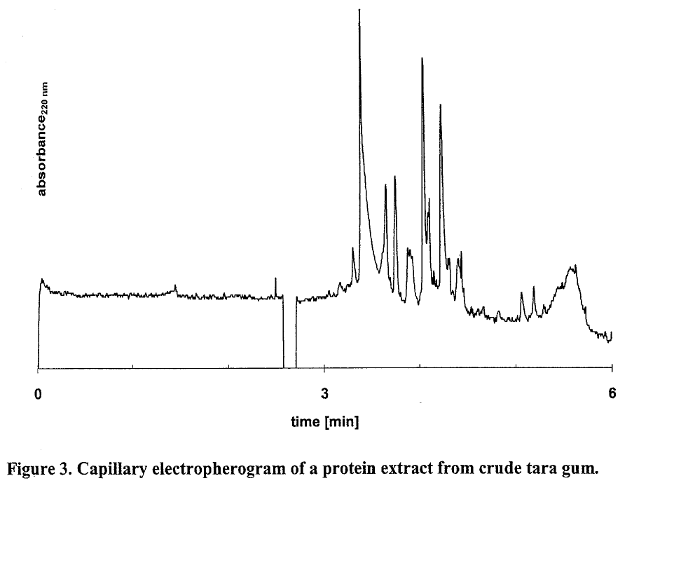

FIG. 4 is capillary electropherogram of proteins from guaran (Jaguar 2229) purified by method 3 (of example 3, below), sampled electrokinetically 10 s at 3.0 kV.

FIG. 5 is a scan of gel from slab gel SDS electrophoresis of guaran proteins. Lanes 1, 5, 15: Novex Mark 12™ molecular mass standards (from the top: myosin, 200 k; β-galactosidase, 116 k; phosphorylase b, 97 k; BSA, 66.3 k; glutamic anhydrase, 55.4 k; lactate dehydrogenate, 36.5 k; carbonic anhydrase, 31 k; trypsin inhibitor, 21.5 k; lysozyme 14.4 k; aprotinin 6 k; insulin B, 3.5 k; insulin A, 2.5 k); lane 2: 4 μL 1 ng/mL BSA; lane 3: 2 μL 1 ng/mL BSA; lane 4: 1 μL 1 ng/mL BSA; lanes 6: 5 μL crude 0.5% Jaguar 2229; lanes 7,: 3 μL crude 0.5% Jaguar 2229; lanes 8, 15 μL purified 0.5% Jaguar 2229 (Method 3); lanes 9: 10 μL purified 0.5% Jaguar 2229 (Method 3); lane 10: 5 μL 67% acetonitrile extracts from crude Jaguar 2229 conc 10×, dil. 10× in SDS-Tris-Glycine buffer; lane 11: 3 μL 67% acetonitrile extracts from crude Jaguar 2229 conc 10×, dil. 10× in SDS-Tris-Glycine buffer; lane 12: 1 μL 67% acetonitrile extracts from crude Jaguar 2229 conc 10×, dil. 10× in in SDS-Tris-Glycine buffer; lane 13: 5 μL 67% acetonitrile extracts from crude Jaguar 2229 conc 10×, dil. 10× in Novex sample buffer; lane 14: 3 μL 67% acetonitrile extracts from crude Jaguar 2229 conc 10×, dil. 10× in Novex sample buffer.

FIG. 6 is a Debye plot and SEC chromatogram of purified guaran (Jaguar 2229).

FIG. 7 is a detail of capillary electropherogram of T-rack DNA sequencing fragments from M13.

FIG. 8 is a capillary electropherogram of DNA sequencing fragments from M13 with 15 g/l guaran as a sieving matrix.

FIG. 9 is a SDS capillary electropherogram of model proteins.(bovine milk α-lactalbumin, 14.2 k; bovine erythrocyte carbonic anhydrase, 31 k; ovalbumin, 45 k; bovine serum albumin (BSA), 66.2 k; rabbit muscle phosphorylase b, 97 k; E. coli β-galactosidase, 116 k) in 10 g/L guaran (M_{w} 7.7×105) as a sieving matrix with UV detection.

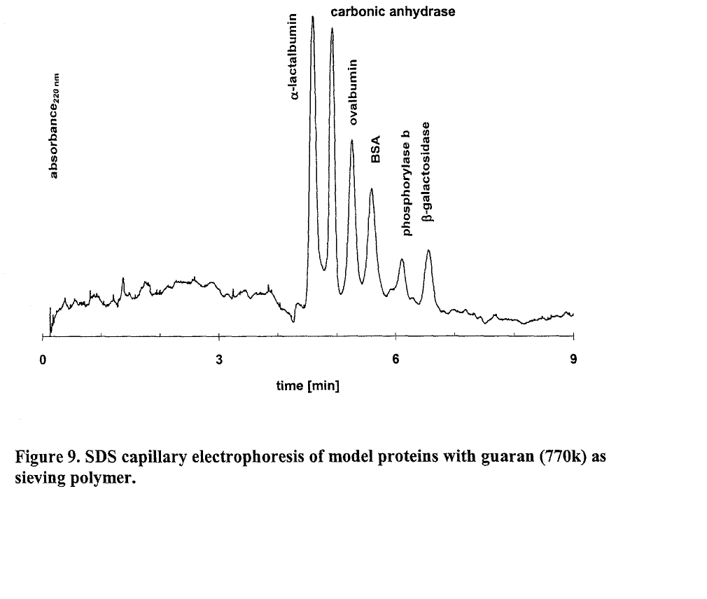

FIG. 10 is a plot of log molecular mass of the model proteins vs. mobility calculated from the run shown in FIG. 9.

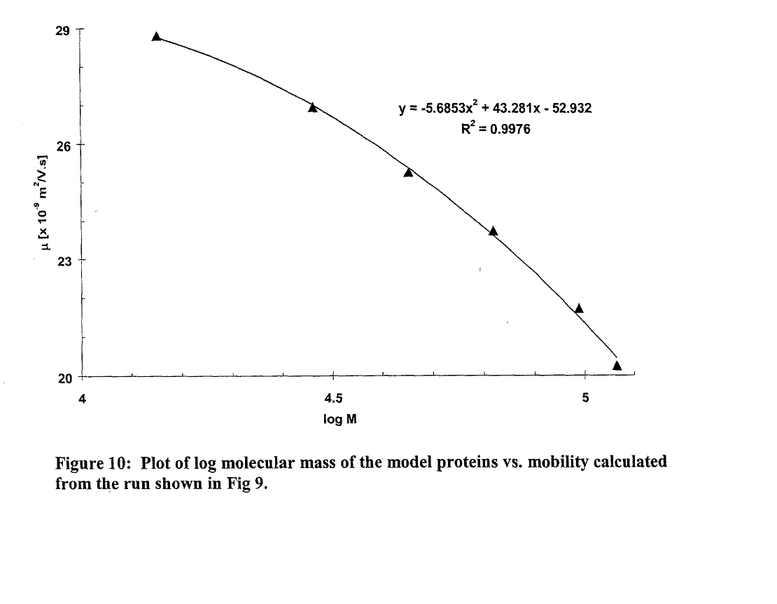

FIG. 11 is a SDS capillary electropherogram of low molecular mass FITC labeled proteins (bovine lung aprotinin, 6, 5 k; bovine milk (α-lactalbumin, 14.2 k; soybean trypsin inhibitor, 20.1 k; bovine erythrocyte carbonic anhydrase, 29 k; horse liver alcohol dehydrogenase, 39.8 k) in 10 g/L guaran (M_{w} 2.2×10⁶) as a sieving matrix with laser induced fluorescence detection.

### DETAILED DESCRIPTION OF THE INVENTION

Sieving properties of galactomannans depend strongly on their molecular mass. We analyzed galactomannans for their weight average molecular mass (M_{w}) and found guar gum to have weight average molecular mass typically over 2 million, whereas tara gum and locust bean gum have molecular mass about 1 million (see Table 1).

TABLE 1
Weight average molecular mass of crude galactomannans

| Preparation | M_{w} [×10⁶] |
|---|---|
| Jaguar 2229 batch H9906527H (Rhodia) | 2.50 |
| Jaguar HV400F (Rhodia) | 2.29 |
| Jaguar 8012 (Rhodia) | 2.10 |
| Jaguar 8009 (Rhodia) | 2.26 |
| Jaguar 8000 (Rhodia) | 2.27 |
| Jaguar A2s (Celanese) | 0.92 |
| Supercol G3-NF (Hercules) | 1.85 |
| Supercol U-NF (Hercules) | 2.07 |
| Guar gum batch 95H0653 (Sigma) | 2.04 |
| Guar gum batch 47H1452 (Sigma) | 2.00 |
| Guar gum (Carbomer) | 2.02 |
| Locust bean gum (Sigma) | 1.12 |
| Locust bean gum Frutosone (Meer) | 0.68 |
| Locust bean gum Synergel | 0.15 |
| Tara gum (Bunge Foods) | 1.02 |

Disclosed herein are capillary columns containing composition made of galactomannan, buffer, denaturant, and detergent. In a preferred embodiment the galactomannan is guar gum, tara gum, or locust bean gum and concentration of the galactomannan is between 8 g/l and 20 g/l. The composition is especially useful for capillary electrophoresis of DNA sequencing fragments and for SDS gel capillary electrophoresis of proteins. In DNA sequencing the weight average molecular mass (M_{w}) of galactomannans is approximately in the range of 8×10⁵ and 3×10⁶. In SDS gel capillary electrophoresis the weight average molecular mass of galactomannans is approximately in the range of 10⁵ and 3×10⁶. Galactomannans having a molecular weight of at least 300,000 are the preferred choice for sieving matrixes. The viscosity and weight average molecular mass of galactomannans can be reduced by the methods of ultrasonic treatment, autoclaving, acid hydrolysis, and basic hydrolysis. A favorable characteristic of galactomannans is that they are non-toxic and biodegradable.

One preferred capillary column has an interior cavity filled with a composition made of an aqueous solution of 15 g/l guaran, 7M urea, 50 mM TRIS, and 50 mM HEPES. Another preferred capillary column has an interior cavity filled with a composition made of an aqueous solution of 10 g/l guaran having molecular mass of 7.7×10⁵, 50 mM TRIS, 50 mM HEPES, and 4 mM SDS (sodium dodecyl sulfate).

Desirable buffers include buffers that are UV transparent and buffers wherein, the pKₐ of the base and acid do not differ by more than 1.0 and wherein the pKₐ of the first ionization degree of said base and said acid is in the range from 6 to 9. Desirable buffers also include Tris, N-hydroxyethyl-mopholine, N-ethylmorpholine, Ammediol, HEPES, Tricine. A desirable molarity for the buffer is 0.1M.

Desirable detergents include: sodium dodecyl sulphate, lithium dodecyl sulphate, cetyltrimethylammonium bromide, cetyltrimethylammonium chloride, pyridinium bromide, pyridinium chloride, and carboethopendecenium bromide. Desirable denaturants include: urea, dimethylsulfoxide, formamide, monomethylformamide, dimethylformamide, ethylformamide, diethylformamide, and methylethylformamide.

Desirable anion exchange resins include: crosslinked dextran based QAE Sephadex A-25 and polystyrene based Source 30Q. Desirable cation exchange resins include: crosslinked dextran based SP Sephadex C-25 and polystyrene based Source 30S.

Desirable detection methods for use with capillary columns and DNA and protein analysis include: UV absorption detection, laser-induced flouorescence detection, amperometric detection, conductometric detection and radiometric detection.

Galactomannans contain relatively high content of proteins. Some of the proteins are basic with pI above 9. At the pH of capillary electrophoresis of DNA when galactomannans can be potentially used as a sieving matrix, those proteins behave as cations that interact with DNA by strong Coulombic interactions. These interactions lead to a peak broadening and loss of resolution. Because of that, the protein content has to be minimized in galactomannans that are to be used as sieving matrix. Generally speaking we apply three or four steps to remove proteins from the galactomannan preparations: (i) hydrolysis with NaOH, if it is desirable to reduce the molecular weight of the galactomannan, (ii) treatment with cation exchanger, (iii) treatment with anion exchanger, (iv) precipitation of galactomannan with acetone, which can be repeated if desired. Precipitation in acetone both concentrates the guaran from dilute solutions and fractionates because, the larger molecules are precipitated. The specific purification methods 1-3, shown in Examples 1-3 below, combine these individual steps.

DNA analysis can be performed using the preferred capillary column having an interior cavity filled with a composition made of an aqueous solution of 15 g/l guaran, 7M urea, 50 mM TRIS, and 50 mM HEPES, by introducing an aliquot of a sample into the capillary column, applying an electric field of about 150 V/cm to the capillary column, separating the sample components and detecting them.

Protein analysis can be performed using the preferred capillary column having an interior cavity filled with a composition made of an aqueous solution of 10 g/l guaran having molecular mass of 7.7×10⁵, 50 mM TRIS, 50 mM HEPES, and 4 mM SDS, by introducing an aliquot of a sample into the capillary column, applying an electric field of about 400 V/cm to the capillary column, separating the sample components and detecting them.

TABLE 2
Comparison of guaran purified by the described purification methods

| | Method 1 | Method 2 | Method 3 |
|---|---|---|---|
| | (of Ex. 1) | (of Ex. 2) | (of Ex. 3) |
| Protein content (CZE) | 0.02% | 0.003% | 0.001% |
| Protein content (fluorimetry) | 0.042% | 0.025% | 0.017% |
| Yield | 35% | 45% | 24.5% |
| Molecular mass of guaran | 1.4 × 10⁶ | 1.8 × 10⁶ | 2.2 × 10⁶ |

### EXAMPLES

The following examples are for illustration purposes only and should not be used in any way to limit the appended claims.

### Example 1

### Purification of Galactomannan with NaOH Treatment and Precipitation (Method 1)

8 g/L aqueous solution of guaran was centrifuged at 6,000 RPM for 60 minutes and filtered through 47 mm syringe prefilters (Millipore, Bedford, Mass.). After adding NaOH to final concentration of 0.2 M, the solution was heated to 85-95° C. for and cooled to 50-55° C. Glacial acetic acid was added to neutralize the reaction. Guaran was precipitated with the addition of 2× volume of acetone. The precipitate was washed with acetone and dried at 50° C. The dry guaran was dissolved in water to make 6-8 g/L solution and stirred with Amberlite MB-150. After removal of the resin by filtration, guaran was isolated by precipitation with 2× volume of acetone and drying at 50° C.

### Example 2

### Purification of Galactomannan by Stirring with Dextran Ion Exchangers (Method 2)

6 g/L aqueous solution of guaran in 50 mM N-methylglucamine (MGA)-HCl, pH 9.5 was centrifuged at 6,000 RPM for 60 minutes and filtered through 47 mm syringe prefilters. The filtered solution was mixed with a one-half volume of QAE Sephadex A-25 and stirred on a rotator overnight. The solution was filtered and a second half volume of QAE Sephadex A-25 was added and the process was repeated a second time. The solution was deionized with Amberlite MB-150. 0.5 M sodium acetate buffer, pH 4 was added to give a 50 mM sodium acetate guaran solution. A half volume of SP Sephadex C-25 was added to the acetate buffered guaran solution and the suspension was stirred overnight on a rotator. The solution was filtered and the process repeated with a second half volume of SP Sephadex C-25. The guaran solution was deionized with Amberlite MB-150. Purified guaran was isolated by precipitation with a 2× volume of acetone, an acetone wash 2×, and drying at 50° C.

### Example 3

### Purification of Galactomannan with Polystyrene Based Ion Exchangers (Method 3)

8 g/L aqueous solution of guaran in 25 mM MGA-HCl, pH 9.5 was centrifuged at 6,000 RPM for 90 minutes. After decanting, the solution was diluted to 4 g/L with MGA-HCl buffer and vacuum filtered through Whatman No. 3. The guaran solution was passed through a fritted disc Buchner funnel containing one quarter volume of Source 30Q (Amersham Pharmacia Biotech). The solution was deionized with Amberlite MB-150. Sodium acetate was added to the guaran solution to make 25 mM sodium acetate, pH 5. This solution was passed through a fritted disc Buchner funnel containing one quarter volume of Source 30S. The solution was deionized with Amberlite MB-150. The guaran was isolated by precipitation with a 2× volume of acetone, acetone wash 2×, and drying at 50° C.

### Example 4

### Capillary Electrophoresis of Proteins from Galactomannans

50 mg galactomannan (purified according to method 3, as shown in Example 3 above) was shaken with 200 μL acetonitrile and 100 μL water for 5 min in 1.7 mL Eppendorf tube. The suspension was centrifuged for 1 min at 10,000 RPM in a regular bench top centrifuge and supernatant was collected and used for analysis. For quantitative analysis of proteins in crude sample, 100 μL sample was transferred into 0.6 mL Eppendorf tube and evaporated under vacuum. The residuum on the bottom of the tube was dissolved in 100 μL 50 mM methylglucamine (MGA)-50 mM ε-aminocaproic acid (EACA). For quantitative analysis of proteins in purified guaran, 500 mg guaran was mixed with 2.0 mL water and 4.0 mL acetonitrile. The suspension was shaken for 30 minutes and centrifuged for 1 min at 7,000 RPM. Supernatant with a measured volume was freeze dried in 15 mL tubes. The evaporate was dissolved in 0.5 mL of water, transferred into 0.6 mL Eppendorf tube and freeze dried again. The evaporate at the bottom of the tube was dissolved in 10 μL 50 mM MGA-EACA.

The capillary zone electrophoresis of proteins from galactomannans was performed in a prototype instrument consisting of power supply CZE1000R (Spellman, Plainview, N.Y.), and Spectra 100 UV-Vis detector (Thermo Separation Products, Riviera Beach, Fla.). The separation was performed in a bare fused silica capillary (Polymicro Technologies, Phoenix, Ariz.) 50 cm long, 40 cm effective length (75 μm ID, 360 μm OD). Before analysis the capillary was rinsed with 0.1 M NaOH, deionized water, and background electrolyte consisting of 50 mm methylglucamine (MGA), 50 mm ε-aminocaproic acid (EACA). The capillary was filled with the background electrolyte for the analysis. The samples were injected either by siphoning for 3 s or electrokinetically for 10 s at 3 kV. Separation was performed in 50 mM MGA, 50 mM EACA at 20 kV, proteins were detected by measuring absorbance at 220 nm. The signal was collected by LabPC+ via Measure for Windows (both National Instruments, Austin, Tex.) and processed in Excel 97 (Microsoft, Redmond, Wash.).

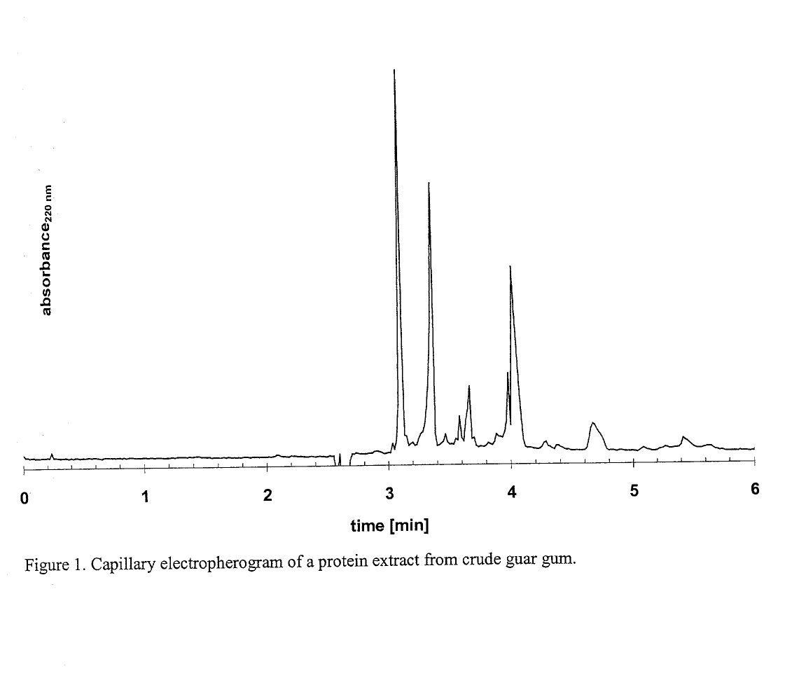

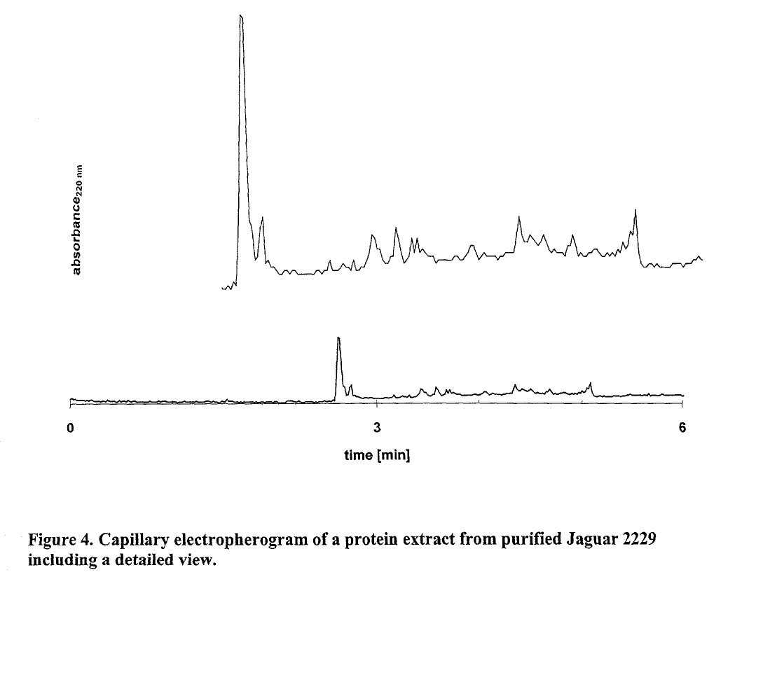

To quantitate proteins in individual fractions of guaran extracts, bovine serum albumin (BSA) monomer was used as a standard. 1-6 mg/mL standard solutions in 50 mM MGA, 50 mM EACA were prepared. The standards and samples were injected electrokinetically for 10 s at 10 kV. The peak areas were corrected to mobility differences between standard and the peak in question. Results can be seen in FIG. 4, demonstrating the absence of protein peaks observed in the electropherogram for crude guar gum, as shown in FIG. 1.

### Example 5

### SDS Slab Gel Electrophoresis of Proteins from Galactomannan

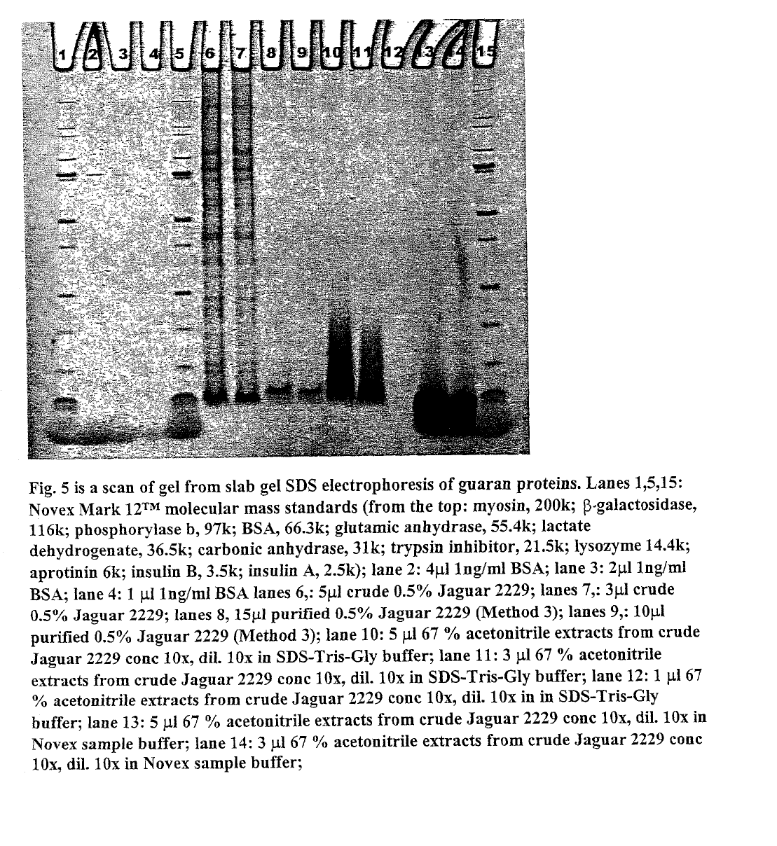

Sodium dodecyl sulfate (SDS) slab gel electrophoresis was performed in Novex Cell II Mini-Cell electrophoresis tank with Novel 10-20% Tris-Glycine polyacrylamide gels and 0.5% SDS Tris-glycine buffer (all Invitrogen, San Diego, Calif.). In general terms the protocol recommended by Novex was followed during the procedure. Guaran was dissolved in 1 g/L concentration and mixed with 2×SDS buffer to make samples. These samples were injected in volumes of 5-10 μL. Sixty seven percent (67%) acetonitrile extracts from guar gum were evaporated to 1/10 volume and diluted with 2×SDS sample buffer. The acetonitrile extracts were injected in volumes of 1-3 μL. The separation was performed at 75 V for 15 min followed by 100 V for 90 min. After the separation the gels were stained with Sypro Red (Molecular Probes, Eugene, Oreg.) and scanned with Fluorlmager 595 (Molecular Dynamics, APB, Sunnyvale, Calif.) with 514 nm excitation filter and 590 nm emission filter. Results can be seen in FIG. 5.

### Example 6

### Determination of Molecular Mass of Polymer (Guaran, Jaguar 2229, Purified by Method 3)

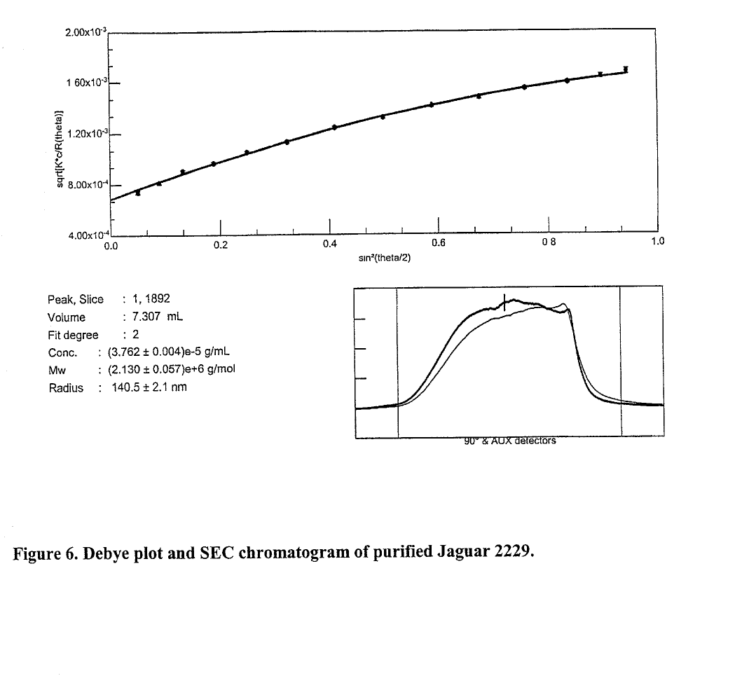

The molecular mass of the sieving polymer was determined by size-exclusion chromatography (SEC) with multiple-angle laser light scattering (MALLS) detection. An Alliance HPLC chromatographic system with an Ultrahydrogel™ Linear column and refractometric detector Waters 2410 (all Waters, Milford, Mass.) and MALLS detector DAWN (Wyatt, Santa Barbara, Calif.) were used to perform size exclusion chromatography with 0.1 M NaNO₃, 0.5 g/L NaN₃ as the mobile phase using flow rate of 0.2 mL/min. ASTRA software (Wyatt, Santa Barbara, Calif.) was used to evaluate the experimental data. Results can be seen in FIG. 6.

### Example 7

### Sieving Matrix for DNA Sequencing Containing Guaran

Solution comprising 15 g/L purified guaran (M_{w} 2.2×10⁶), 7 M urea, 25 mM Tris, 25 mM N-[2-hydroxyethyl]piperazine-N′-2-ethanesulfonic acid (HEPES) in deionized water is prepared by dissolving 1.50 g guaran purified by Method 1-3 (any of these methods) in 95.0 mL 7.37 M urea under stirring at room temperature. When all guaran is dissolved 5.0 mL 0.50 M Tris, 0.50 M HEPES is added and the mixture is stirred for several more hours to make a homogenous solution.

### Example 8

### Sieving Matrix for DNA Sequencing Containing Locust Bean Gum

Solution comprising 10 g/L purified locust bean gum (M_{w} 10⁶), 7 M urea, 25 mM Tris, 25 mM HEPES in deionized water. It is prepared by extremely slow addition of 1.00 g locust bean gum purified by Method 1-3 to 60.5 mL water heated to 95° C. under stirring. When all locust bean gum is disolved the solution is cooled down to room temperature. 42.0 g urea and 5.0 mL 0.50 M Tris, 0.50 M HEPES is added and the mixture is stirred for several hours to make a homogenous solution.

### Example 9

### Sieving Matrix for DNA Sequencing Containing Tara Gum

Solution comprising 12 g/l purified tara gum (M_{w} 10⁶), 7 M urea, 25 mM Tris, 25 mM HEPES in deionized water is prepared by dissolving 1.20 g tara gum purified by Method 1-3 in 95 mL 7.37 M urea under stirring at room temperature. When all tara gum is dissolved 5.0 mL 0.50 M Tris, 0.50 M HEPES is added and the mixture is stirred for several more hours to make a homogenous solution.

### Example 10

### Sieving Matrix Containing Guaran for Manually Operated Capillary SDS Gel Electrophoresis

Solution comprising 10 g/l purified guar gum (M_{w}, 7×10⁵), 50 mM Tris, 50 mM HEPES, 4 mM SDS in deionized water is prepared by dissolving 1.0 g guaran purified by Method 1-3 in 100 mL 50 mM Tris, 50 mM HEPES, 4 mM SDS 50 under stirring at room temperature. Guaran has to be added extremely slowly to ensure homogenous solution. When all guaran is dissolved the solution is spun at 10,000 RPM for 10 min to remove bubbles. To reduce molecular mass of guaran, the solution is autoclaved at 120° C. for 40 min and treated with ultrasound for 3 hrs.

### Example 11

### Sieving Matrix Containing Guaran for Instrument Operated Capillary SDS Gel Electrophoresis

Solution comprising 10 g/l purified guar gum (M_{w}, 2.2×10⁶), 50 mM Tris, 50 mM HEPES, 4 mM SDS in deionized water is prepared by dissolving 1.0 g guaran purified by Method 1-3 in 100 mL 50 mM Tris, 50 mM HEPES, 4 mM SDS 50 under stirring at room temperature. Guaran has to be added extremely slowly to ensure homogenous solution. When all guaran is dissolved the solution is spun at 10,000 RPM for 10 min to remove bubbles.

### Example 12

### DNA Sequencing by Capillary Electrophoresis

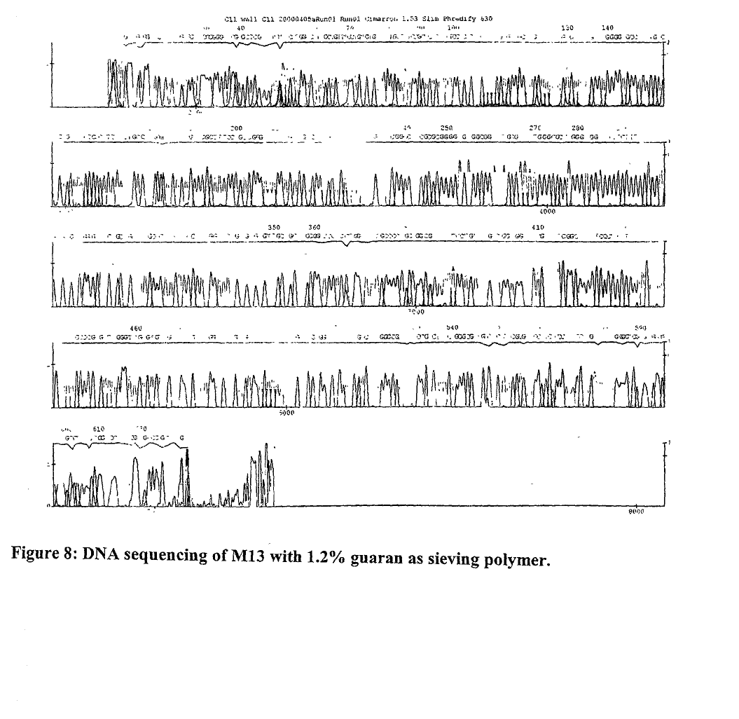

DNA sequencing was performed on the instrument for capillary array electrophoresis MegaBACE 1000™ (Molecular Dynamics, Sunnyvale, Calif.). The instrument is equipped with 96 capillaries organized in 6 arrays The fused silica capillaries are 62 cm long with the effective length of 40 cm, internal diameter of 75 μm, and outer diameter of 360 μm. The instrument was thermostated at 44° C. Before a DNA sequencing run the capillaries were twice flushed with deionized water at pressure of 620 kPa (90 psi). Guaran sieving matrix according to Example 7 was delivered into 2 mL tubes and centrifuged at 10,000 RPM to remove bubbles. The tubes with the guaran matrix were placed in the anode stage and pumped under pressure 6.9 MPa (1000 psi) for approx. 120 s. 250 μL PCR tubes were filled with 200 μL guar sieving matrix, placed in a 96 tube rack, and spun at 4,000 RPM in a centrifuge with a micro titer plate holder to remove bubbles. Guaran matrix was left for 10 minutes to relax and renew its random structure. Relaxation was followed by an electrophoretic pre-run for 10 minutes at 4 kV. Before sample injection the capillary tips were extensively washed with deionized water to remove guaran matrix from the outer surface of the capillaries. M13 DNA sequencing fragments were injected electrokinetically for 30 s at 4 kV. After the injection the capillary tips were washed again to remove excessive sample. The electrophoretic run was made applying voltage of +10 kV on the anode side of the system for 90 min. The DNA fragments were detected by laser induced fluorescence and the collected data were evaluated with a base caller (Molecular Dynamics, Sunnyvale, Calif.) to determine read length. After the electrophoretic run, the sieving matrix was pumped out under pressure of 6.9 MPa (1000 psi) and flushed repeatedly with distilled water under pressure of 620 kPa (90 psi). Results are shown in FIG. 8.

### Example 13

### One-color DNA Sequencing

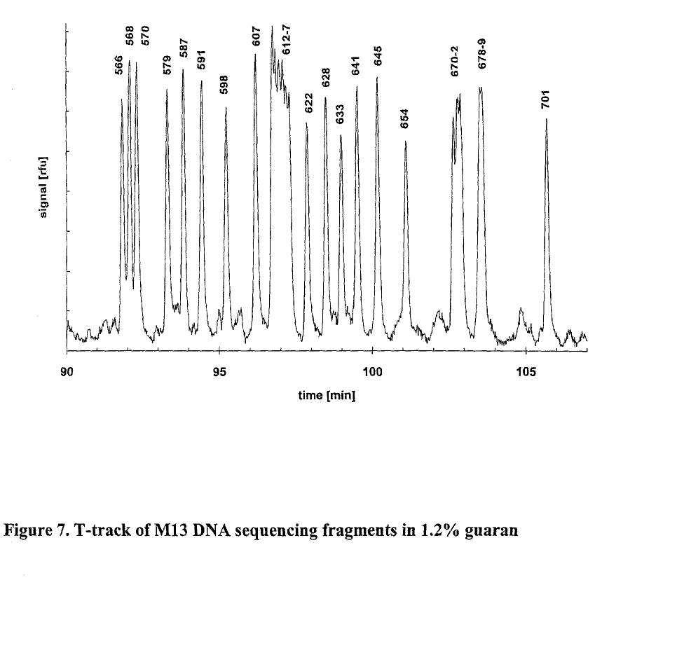

Separation of one-color DNA sequencing fragments (T-rack) was performed on a breadboard at room temperature in capillaries (75 μm i.d., 200 μm o.d.) of the total length 65 cm and effective length 40 cm coated with linear polyacrylamide [Dolnik, V. et al., J. Microcol. Sep. 1998, 10, 175]. The sieving matrix was prepared as described in Example 7. The T-track M13 samples were injected electrokinetically for 20 s at 6 kV. The separation was performed at 12 kV for 70 min. Laser light from 20 mW argon laser (488 nm) was used to emit fluorescence that detection signal was collected at 530 nm with the frequency 1 Hz for each capillary. The data were analyzed using ArrayQuant™ software (Molecular Dynamics, Sunnyvale, Calif.). Results are shown in FIG. 7.

### Example 14

### Capillary SDS Gel Electrophoresis of Proteins with Manually Operation and UV Detection

The capillary zone electrophoresis of proteins was performed in a prototype instrument consisting of power supply CZE1000R (Spellman, Plainview, N.Y.), and Spectra 100 UV-Vis detector (Thermo Separation Products, Riviera Beach, Fla.). The separation was performed in a fused silica capillary (Polymicro Technologies, Phoenix, Ariz.) 40 cm long, 30 cm effective length (75 μm ID, 360 μm OD) coated with linear polyacrylamide [Cobb, K. A. et al., Anal. Chem. 1990, 62, 2478, Dolnik, V. et al., J Microcol. Sep. 1998, 10, 175]. Before analysis the capillary was rinsed with deionized water and 0.1 M Tris, HEPES, 4 mM SDS and filled with the sieving matrix from Example 10. The samples were injected electrokinetically for 20 s at −15 kV. Separation was performed at −15 kV, proteins were detected by measuring absorbance at 220 nm. The signal was collected by LabPC+ via Measure for Windows (both National Instruments, Austin, Tex.) and processed in Excel 97 (Microsoft, Redmond, Wash.).

### Example 15

### Capillary SDS Gel Electrophoresis of Proteins with High Pressure Capillary Filling and Laser Induced Fluorescence Detection

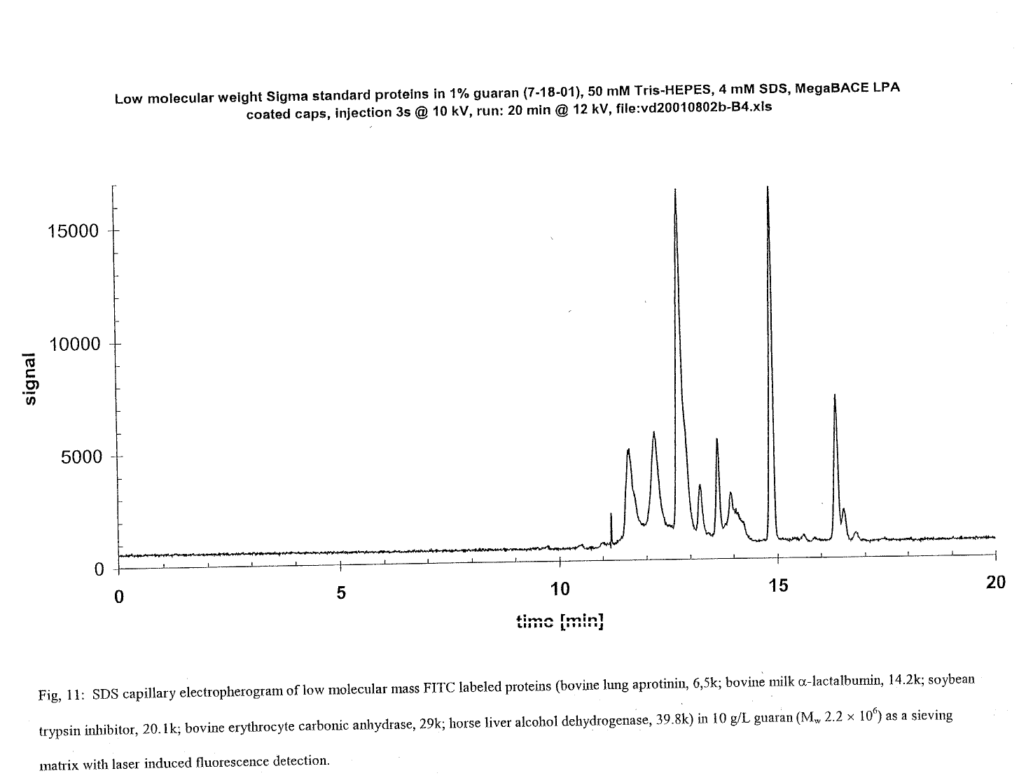

SDS capillary electrophoresis was performed on the instrument for capillary array electrophoresis MegaBACE 1000™ (Molecular Dynamics, Sunnyvale, Calif.). The instrument is equipped with 96 capillaries organized in 6 arrays The fused silica capillaries are 62 cm long with the effective length of 40 cm, internal diameter of 75 μm, and outer diameter of 360 μm. The instrument was thermostated at 27° C. Before a separation run the capillaries were twice flushed with deionized water at pressure of 620 (kPa (90 psi). Guaran sieving matrix made after Example 11 was delivered into 2 mL tubes and centrifuged at 10,000 RPM to remove bubbles. The tubes with the guaran matrix were placed in the anode stage and pumped under pressure 6.9 MPa (1000 psi) for approx. 30 s. 96 micro well plate was filled with 100 μL 50 mM Tris, 50 mM HEPES, 4 mM SDS. Before sample injection the capillary tips were extensively washed with deionized water to remove guaran matrix from the outer surface of the capillaries. FITC labeled proteins were injected for 3 s at 10 kV. After the injection the capillary tips were washed again to remove excessive sample. The electrophoretic run was made applying voltage of +12 kV on the anode side of the system for 20 min. The FITC labeled proteins were detected by measuring fluorescence induced with 488 nm argon laser. After the electrophoretic run, the sieving matrix was pumped out under pressure of 6.9 MPa (1000 psi) and flushed repeatedly with distilled water under pressure of 620 kPa (90 psi). Results shown in FIG. 11.

Those skilled in the art having the benefit of the teachings of the present invention as set forth above, can effect numerous modifications thereto. These modifications are to be construed as being encompassed within the scope of the present invention as set forth in the appended claims.

What is claimed is:

1. A composition comprising:
a galactomannan having 50-90% by weight of mannose residues;
10-50% by weight of galactose residues; and
less than 0.1% by weight of protein.

2. The composition of claim 1 wherein, the galactomannan is guar gum of Cyamopsis tetragonoluba.

3. The composition of claim 1 wherein, the galactomannan is tara gum of Caesalpinia spinosa.

4. The composition of claim 1 wherein, the galactomannan is locust bean gum of Ceratonia siliqua.

5. The composition of claim 1 wherein, the galactomannan has a weight molecular mass of 3×10⁵ to 4×10⁶.

6. A process of purifying a galactomannan said process comprising:
treating the galactomannan with a anion exchanger;
treating the galactomannan with a cation exchanger;
precipitating the galactomannan with an organic solvent, said organic solvent being selected from the group consisting of ethers, esters, ketones and aldehydes.

7. The process of claim 6 wherein, said organic solvent is acetone.

8. The process of claim 6 further comprising treating said galactomannan with a diluted aqueous solution of base.

9. The process of claim 8 wherein said base is an alkali hydroxide, selected from the group consisting of sodium hydroxide, potassium hydroxide and lithium hydroxide.

10. The process of claim 9 wherein said alkali hydroxide is in the concentration of 0.001 M to 1.0 M.

11. The process of claim 6 wherein said treating steps are performed by ion exchange chromatography.

12. The process of claim 6 wherein said treating steps are performed by mixing and stirring said galactomannan with an anion exchange resin and separately, with a cation exchange resin.

13. The process of claim 12 wherein one or both of said anion exchange resin and said cation exchange resin is a polystyrene resin.

14. The process of claim 12 wherein one or both of said anion exchange resin and said cation exchange resin is a crosslinked dextran resin.

15. The process of claim 12 wherein said anion exchanger resin is QAE Sephadex or Source 30Q.

16. The process of claim 12 wherein said cation exchanger resin is SP Sephadex or Source 30S.

17. The process of claim 6 further comprising treating said galactomannan with a mixed ion exchanger.

18. A capillary column comprising:
a capillary electrophoresis tube, and
contained within said tube, a composition comprising
an aqueous solution containing 0.5 to 3.% by weight of a galactomannan according to claim 1;
a buffer having a concentration of 0.01 M to 0.1 M;
a denaturant having a concentration of 0.0 M to 8.0 M; and
a detergent having a concentration of 0.0 to 1.% by weight.

19. The capillary column of claim 18 wherein, said galactomannan is guar gum of Cyamopsis tetragonoluba.

20. The capillary column of claim 18 wherein, said galactomannan is tara gum of Caesalpinia spinosa.

21. The capillary column of claim 18 wherein, said galactomannan is locust bean gum Ceratonia siliqua.

22. The capillary column of claim 18 wherein, said galactomannan has a weight average molecular mass of 3×10⁵ to 4×10⁶.

23. The capillary column of claim 18 wherein, said detergent is selected from the group consisting of sodium dodecyl sulphate, lithium dodecyl sulphate, cetyltrimethylammonium bromide, cetyltrimethylammonium chloride, pyridinium bromide, pyridinium chloride, and carboethopendecenium bromide.
